(19) United States
(12) Patent Application Publication
(10) Pub. No.: US 2010/0268285 A1
(43) Pub. Date: Oct. 21, 2010

(54) BONE SCREW SYSTEM AND METHOD FOR THE FIXATION OF BONE FRACTURES

(75) Inventors: Kishore Tipirneni, Glendale, AZ (US); Wayne Vassello, Lake Worth, FL (US)

Correspondence Address: SNELL & WILMER L.L.P. (Main), 400 EAST VAN BUREN, ONE ARIZONA CENTER, PHOENIX, AZ, 85004-2202, US

(73) Assignee: ORTHOIP, LLC, Boca Raton, FL (US)

(21) Appl. No.: 12/769,529

(22) Filed: Apr. 28, 2010

Related U.S. Application Data
(63) Continuation-in-part of application No. 12/425,225, filed on Apr. 16, 2009.
Application No. 12/425,225 is a continuation-in-part of application No. 12/369,589, filed on Feb. 11, 2009.
Application No. 12/369,589 is a continuation-in-part of application No. 12/258,013, filed on Oct. 24, 2008.
Application No. 12/258,013 is a continuation-in-part of application No. 12/104,658, filed on Apr. 17, 2008.
Application No. 12/104,658 is a continuation-in-part of application No. 11/952,715, filed on Dec. 7, 2007.
Application No. 11/952,715 is a continuation-in-part of application No. 11/742,457, filed on Apr. 30, 2007.
Application No. 11/742,457 is a continuation-in-part of application No. 11/678,473, filed on Feb. 23, 2007.
Application No. 11/678,473 is a continuation-in-part of application No. 10/779,892, filed on Feb. 17, 2004, now Pat. No. 7,591,823.
Application No. 10/779,892 is a continuation of application No. 10/272,773, filed on Oct. 17, 2002, now Pat. No. 6,736,819.
(60) Provisional application No. 60/330,187, filed on Oct. 18, 2001.

(51) Int. Cl. A61B 17/86; A61B 17/56

(52) U.S. Cl. 606/309; 606/104

(57) ABSTRACT

A bone screw comprising a sleeve and a shaft reciprocally received within the sleeve. The bone screw may be partially or fully extended prior to insertion into a bone by inserting a driver into a longitudinal opening in the sleeve to push the shaft out of the sleeve. The bone screw may be used in connection with a locking plate or other stabilization device. The sleeve of the bone screw may have a smooth distal end. The distal end may have a wider diameter than the main body of the sleeve so as to sit within a counter bore in the locking plate. A threaded set screw may be used to secure the bone screw within the locking plate.

### CROSS-REFERENCE TO RELATED APPLICATIONS

This application is a continuation-in-part of, and claims priority to, U.S. Ser. No. 12/425,225 filed Apr. 16, 2009 and entitled “BONE SCREW SYSTEM AND METHOD FOR THE FIXATION OF BONE FRACTURES.” The '225 application is a continuation-in-part of and claims priority to U.S. Ser. No. 12/369,589 filed Feb. 11, 2009 and entitled “STABILIZATION SYSTEM AND METHOD FOR THE FIXATION OF BONE FRACTURES.” The '589 application is a continuation-in-part of and claims priority to U.S. Ser. No. 12/258,013 filed Oct. 24, 2008 and entitled “BONE SCREW SYSTEM AND METHOD.” The '013 application is a continuation-in-part of and claims priority to U.S. Ser. No. 12/104,658 filed Apr. 17, 2008 and entitled “ADJUSTABLE BONE PLATE FIXATION SYSTEM AND METHOD.” The '658 application is a continuation-in-part of, and claims priority to, U.S. Ser. No. 11/952,715 filed on Dec. 7, 2007, and entitled “BONE SCREW SYSTEM AND METHOD.” The '715 application is a continuation-in-part of, and claims priority to, U.S. Ser. No. 11/742,457 filed on Apr. 30, 2007, and entitled “BONE SCREW SYSTEM AND METHOD.” The '457 application is a continuation-in-part of, and claims priority to, U.S. Ser. No. 11/678,473 filed on Feb. 23, 2007, and entitled “CANNULATED BONE SCREW SYSTEM AND METHOD.” The '473 application is a continuation-in-part of, and claims priority to, U.S. Ser. No. 10/779,892 filed on Feb. 17, 2004, and entitled “SYSTEM AND METHOD FOR THE FIXATION OF BONE FRACTURES.” The '892 application is a continuation of, and claims priority to, U.S. Ser. No. 10/272,773 filed on Oct. 17, 2002, and entitled “SYSTEM AND METHOD FOR THE FIXATION OF BONE FRACTURES” (now U.S. Pat. No. 6,736,819, issued on May 18, 2004). The '819 patent is the non provisional application of, and claims priority to, U.S. Provisional Application Ser. No. 60/330,187 filed on Oct. 18, 2001, and entitled “LAGWIRE SYSTEM AND METHOD.” All of which are incorporated herein by reference in their entirety.

### FIELD OF INVENTION

The invention generally relates to bone screw systems and methods for the fixation of fractures in one or more objects, and more particularly, to medicated bone screws incorporating internal bushings which may fully or partially extend prior to engaging a bone and may be used in connection with a bone stabilization device, such as a locking plate.

### BACKGROUND OF THE INVENTION

It is well-known in the medical arts that constant pressure on a bone fracture speeds healing. As such, orthopedic physicians frequently insert one or more bone screws in the area of the fracture to provide pressure. The bone screws are typically used in connection with one or more bone stabilization devices, such as a locking plate, to provide additional support to the fracture.

Existing bone screws have various disadvantages. For example, the shafts of conventional bone screws are generally not extendable relative to the sleeves until the screw reaches the bone, making it difficult for operators to ascertain how far the shaft should be extended. Moreover, when conventional bone screws are used in connection with locking plates, only limited extension of the shaft can occur before the threads of the locking plate secure with the threads of the bone screw and prevent further extension (e.g., 1 to 2 rotations).

Another disadvantage of conventional bone screws is that they are not readily securable relative to the bone stabilization devices at a specific angle of entry, and thus permit movement of the bone screw relative to the stabilization device.

Accordingly, a need exists for a bone screw device that may be (1) fully or partially extended prior to engaging a bone and/or prior to inserting into a stabilization device; and/or (2) secured to a bone stabilization device at a pre-prescribed angle of entry.

### SUMMARY OF THE INVENTION

In general, the system includes bone screws which facilitate the stabilization and fixation of bone fractures. In an exemplary embodiment, the shaft of the bone screw device may be configured in a fully or partially extended position relative to the sleeve of the bone screw device before engaging the bone.

For example, the sleeve may comprise a longitudinal opening and an instrument (e.g., a driver) which may be inserted into the longitudinal opening to push the shaft of the bone screw into an extended position. In an exemplary embodiment, the bone screw is configured in an extended position and then inserted through a first bone portion into a second bone portion.

In various embodiments, a bone screw does not comprise threads on the distal end but still operably couples with a stabilization device. In an exemplary embodiment, the distal end of the bone screw is of wider diameter than the body of sleeve to allow it to mate with the counter-bore of a locking plate. A threaded set screw is also used to mate with a threaded opening of a locking plate to hold the bone screw in place.

### BRIEF DESCRIPTION OF THE DRAWINGS

A more complete understanding of the present invention may be derived by referring to the detailed description and claims when considered in connection with the figures, wherein like reference numbers refer to similar elements throughout the figures, and:

FIG. 1 illustrates an exploded view of a bone plate system, in accordance with an exemplary embodiment of the present invention;

FIG. 2 illustrates a bone plate system, in accordance with an exemplary embodiment of the present invention;

FIG. 3 is a fixation of a spinal injury in accordance with an exemplary embodiment of the present invention;

FIG. 4 is a cannulated screw having a sleeve and a threaded shaft in accordance with an exemplary embodiment of the present invention;

FIG. 5 is a cannulated screw having a sleeve, a compressive device and a threaded shaft and shown prior to extending the compressive device, in accordance with an exemplary embodiment of the present invention;

FIG. 6 is a cannulated screw having a sleeve, a compressive device and a threaded shaft and shown after extending the compressive device, in accordance with an exemplary embodiment of the present invention;

FIG. 7 is an exploded view of a bone screw incorporating internal bushings;

FIG. 8 is a bone screw having a shaft oriented in an extended position relative to the sleeve, in accordance with an exemplary embodiment of the invention;

FIG. 9 is a bone screw having a longitudinal opening that is engageable by a driver to push the shaft to an extended position, in accordance with an exemplary embodiment of the invention;

FIG. 10 shows multiple cannulated screws providing rotational stability to a fracture, in accordance with an exemplary embodiment of the present invention;

FIG. 11A shows a cannulated screw received through an intermedulary rod, in accordance with an exemplary embodiment of the present invention;

FIG. 11B shows a cannulated screw having an adapter, in accordance with an exemplary embodiment of the present invention;

FIG. 11C shows an adapter having a lip and a plurality of notches mated to a retaining ring on the head of the screw;

FIG. 11D shows a cannulated screw having an adapter mated to the head of the screw;

FIG. 11E shows a cannulated screw received into a locking plate, in accordance with an exemplary embodiment of the present invention;

FIG. 11F shows a bone screw having a non-threaded distal end operable to mate with a counter-bore of a locking plate, in accordance with an exemplary embodiment;

FIG. 11G shows a bone screw received into a locking plate and secured with a set screw, in accordance with an exemplary embodiment;

FIG. 12 shows a cannulated screw with a sleeve and a barrel as part of a hip screw plate system, in accordance with an exemplary embodiment of the present invention;

FIG. 13 shows another embodiment of a cannulated screw wherein the barrel functions as the sleeve, as part of a hip screw plate system, in accordance with an exemplary embodiment of the present invention;

FIG. 14 is a sleeve and a bone screw capable of receding within the sleeve in accordance with an exemplary embodiment of the present invention;

FIG. 15 is a cross section view of the sleeve and bone screw of FIG. 14;

FIG. 16 is a perspective view of the sleeve and bone screw of FIGS. 14 and 15 shown with the bone screw recessed within the sleeve in accordance with an exemplary embodiment of the present invention; and

FIG. 17 is a cross section view of the bone screw recessed within the sleeve of FIG. 16.

### DETAILED DESCRIPTION

The present invention is described herein and includes various exemplary embodiments in sufficient detail to enable those skilled in the art to practice the invention, and it should be understood that other embodiments may be realized without departing from the spirit and scope of the invention. Thus, the following detailed description is presented for purposes of illustration only, and not of limitation, and the scope of the invention is defined solely by the appended claims. The particular implementations shown and described herein are illustrative of the invention and its best mode and are not intended to otherwise limit the scope of the present invention in any way.

In general, the present invention facilitates the change in distance between objects, object portions, or surfaces, compresses objects or object portions together, and/or provides a configurable or random amount of pressure between surfaces. The system may facilitate changing, maintaining, reducing and/or expanding the distance between objects or object portions. The applied pressure may be suitably configured to be constant, increasing, decreasing, variable, random, and/or the like. In an exemplary embodiment, the invention includes a device which may be fixedly or removably attached to pathology, such as to a certain portion of a bone. In a particular embodiment, the device is fixedly or removably attached to the far cortex of the bone. In another embodiment, the invention includes a device or method for retracting the attached device to reduce the distance between the surfaces of the pathology. In a further embodiment, the invention includes a device and/or method for maintaining the pressure between the surfaces of pathology.

In various embodiments, the device may be used in conjunction with systems or components of various other orthopedic devices such as those described in U.S. patent application Ser. No. 12/491,132 ('132), which is incorporated herein by reference in its entirety. In another example, the device may be used in conjunction with support systems such as bone plates.

For example, in an embodiment, a bone plate system may comprise a frame, a track, an insertion niche, one or more fastening plates, and one or more tension members. The frame may be any structure which provides support for the components of a bone plate system. In one embodiment, the center portion of the frame may be configured with a track. The track may be any structure configured to permit fastening plates and tension members to traverse along the length of the bone plate to a desired position.

Fastening plates may be any structure configured to traverse along the length of the track to a desired position and provide support for a fastener, which connects the plate to a bone. Fastening plates may be any suitable size, shape, composition or structure. In one exemplary embodiment, a fastening plate comprises one or more openings adapted to receive a fastener for securing the bone plate to a bone. The openings may be threaded or non-threaded, and may have any suitable size and/or shape, such as circular, square, elliptical, and the like. Moreover, the openings may comprise a counter-bore configured to receive the head of a fastener. In one embodiment, the fastening plate may be configured to rotate (for example, 90 degrees) so as to lock into a desired position along the length of the track.

A fastener may generally comprise any mechanism for securing a bone plate to a bone, including for example a cap, bone screw, lagscrew, lagwire, pin, wire and/or the like. The size of the fastener may be selected based upon the size and shape of the opening of the fastening plate, or vice versa.

A tensioning member may be any structure suitable for providing tension. In one embodiment, tensioning member traverses along the length of the track and compresses axially upon the application of stress. A tensioning member may be, for example, a bias member or spring, such as a coil-spring. In one embodiment, the tensioning member is configured to mate with a fastening plate and provide positional tension. It will be appreciated that any desired number and/or combination of fastening plates and tensioning members may be inserted onto the track of a bone plate system.

An insertion niche may be any structure which permits insertion of one or more fastening plates and/or tension members onto a track of a bone plate. In one embodiment, the insertion niche is located substantially in the center of the bone plate. However, it will be appreciated that the insertion niche may be located at any location on, within or around the bone plate that suitably permits insertion of a fastening plate and/or tension member onto a track.

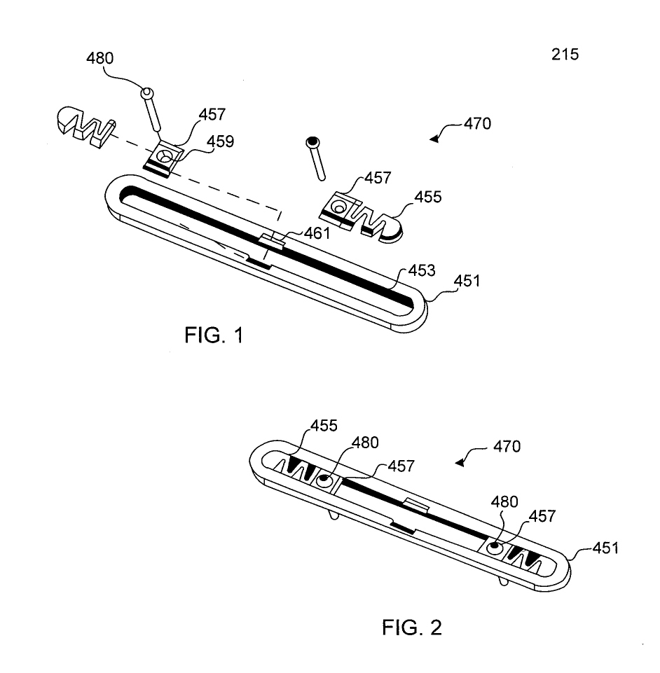

With reference to FIG. 1, an exemplary bone plate system 470 is provided. As shown, frame 451 is substantially oval-shaped and comprises track 453. Track 453 comprises grooves configured to be mated with the grooves of one or more tension members 455 and fastening plates 457. Fastening plate 457 comprises an opening 459 adapted to receive a fastener, such as bone screw 480. Fastening plates 457 and tension members 455 may be inserted onto track 453 via insertion niche 461 in any desired number or configuration. Once inserted, fastening plates 457 may be rotated 90 degrees to lock into sliding position along track 453.

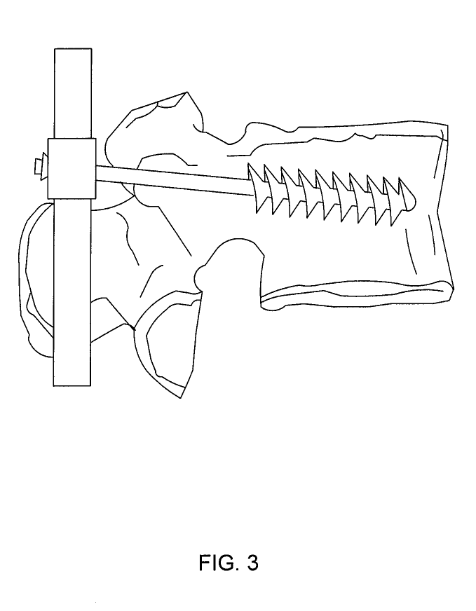

FIG. 2 shows one embodiment of bone plate system 470 comprising two tension members 455 located on either end of frame 451, two fastening plates 457 adjacent to tension members 457, and two fasteners (e.g., bone screws 480).

In accordance with an exemplary method of the present invention, a user may: select a suitable bone plate comprising a track; insert at least one fastening plate and at least one tension member onto the track; slide the fastening plate and the tension member along the track to a desired location; rotate the fastening plate 90 degrees relative to the track to lock the fastening plate into a desired position; and fasten the bone plate to a desired portion using a fastener. It will be understood that various steps provided above may be omitted or performed in any desired order in accordance with the present invention.

It will be understood that bone plates disclosed herein may be any suitable size and shape. For example, a bone plate may be substantially concave, convex, “S”-shaped, “I”-shaped, or “L”-shaped. In an exemplary embodiment, the bone plate is substantially elongate such that the length is greater than the width. Moreover, the size and/or shape of the bone plate may be configured to substantially correspond to the size and shape of the bone and/or conform to the bone being aligned.

Moreover, the bone plates of the present invention may be configured for use on any desired bone, and may comprise any suitable material. In various embodiments, the bone plate may be rigid, and yet flexible so as to conform to a bone. Suitable materials include, for example, stainless steel, various metal alloys, plastics such as PEEK, and various inert materials, among others.

FIG. 3 shows a fixation of a vertebrae in accordance with an exemplary embodiment of the present invention. The screw is inserted into the vertebrae and a cap is fitted onto the end of the wire. The cap is specially constructed such that the cap attaches to a rod. The rod may extend along various vertebrae such that the lagwires may extend from various vertebrae and all connect to the same rod. Another screw and lagwire may be inserted into the other side of the vertebrae such that the wire extends from the other side of the vertebrae and its cap connects to a second rod on the other side of the vertebrae for additional stability.

As described herein, the system and method of the present invention provides a device which is self-drilling, self-tapping and can be inserted under power. The invention also facilitates reducing and fixing fractures in one step. As such, the invention substantially expedites the process for fixation of bone fractures which is, of course, critical during trauma situations in order to stabilize a patient or to minimize the amount of time the patient is on the operating table or under anesthesia. In contrast to typical prior art screws wherein a gliding hole in the near cortex simply guides the screw, the present invention provides the ability for two sides of cortex bone screw fixation. Moreover, because of the strength of the attachment to the bone, the invention enables sufficient fixation even in poor quality bone material. Furthermore, wherein the prior art systems often require the use of cannulated screws in order to utilize a guidewire for placement, the present invention does not require the use of cannulated screws. Because the lagwire includes a tip 4 which creates a pilot hole, taps the bone for threads and fixes the threads into the bone, the system and method minimizes the possibility of inaccurate placement into the distal cortex or missing the distal hole.

In prior art systems, the physician typically cuts a relatively large opening in the skin in order to locate the bone segments, pull the bone segments into alignment, then place the screw into the bones. In the present invention, the system facilitates the percutaneous technique by allowing the physician to cut a minor incision into the skin for the anchor component, insert the anchor component, then pull the bones together with wire 12 and set the cap, all without large incisions or additional incisions.

Another embodiment for a bone fixation device includes a collapsing bone fixation device which is suitably configured to collapse in association with a fracture collapse to minimize or prevent the device from protruding beyond the bone. In an exemplary embodiment, the bone fixation device also includes an internal (i.e., minimal or no contact with the bone) compressive device 140 to maintain compression across the fracture during fracture collapse (e.g., weight bearing by the patient).

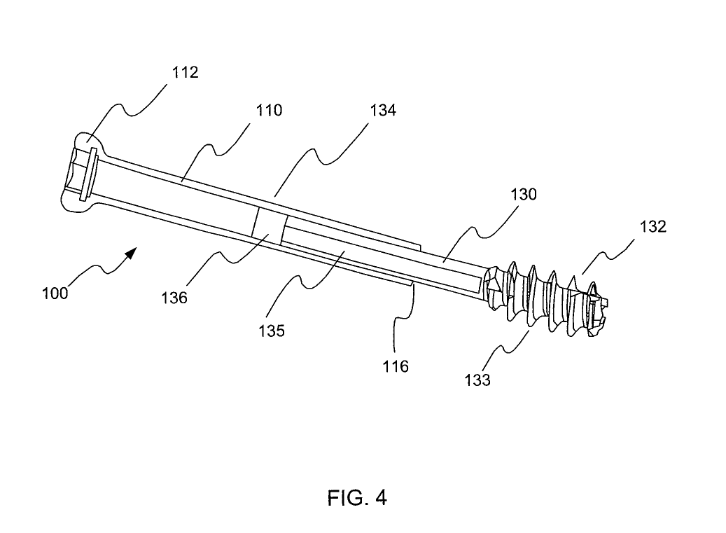

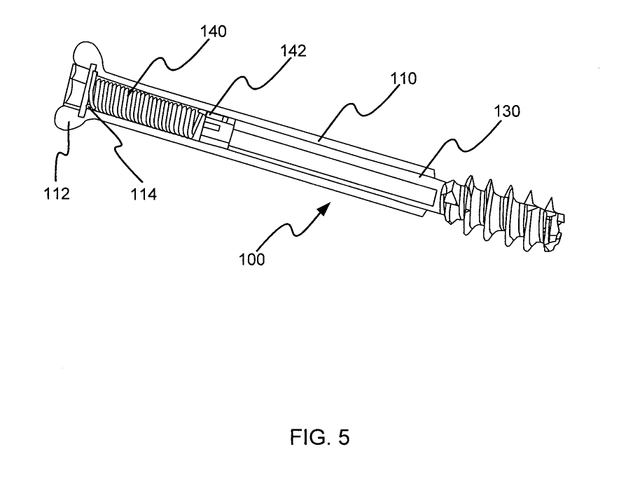

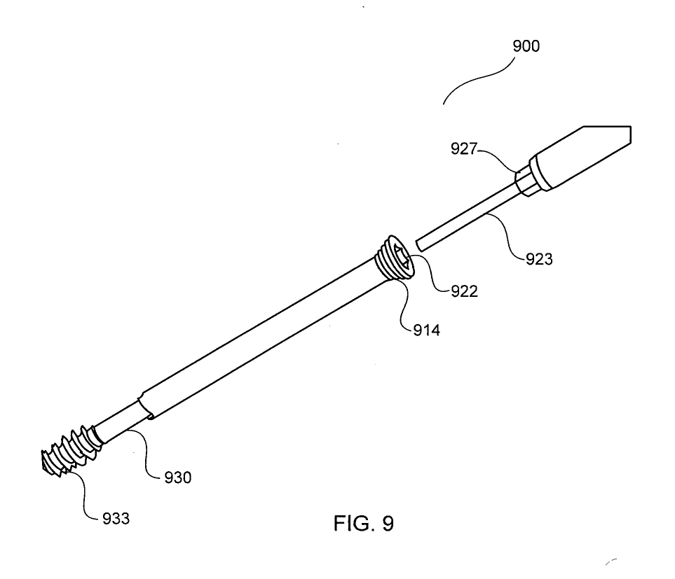

With respect to FIG. 4, an exemplary embodiment includes an improved screw 100 having a sleeve 110 and a shaft 130. In one embodiment, no additional elements exist between sleeve 110 and shaft 130, but in other embodiments (as discussed below in more detail and in FIGS. 5 through 9), a compressive device 140 (e.g. spring) is located between sleeve 110 and shaft 130. In an exemplary embodiment, each of the elements sleeve 110, shaft 130, and compressive device 140 are cannulated.

In one embodiment, with respect to FIG. 4, shaft 130 includes a first end 132 having a gripping device 133 and a second end 134. Gripping device 133 may include any structure and configuration for enabling shaft to enter and attach to an object. In one embodiment, gripping device includes a threaded surface thereon. The threaded surface may include cutting threads, mating threads, barbs, ribbed surface or any other surface configured to retain shaft 130 into an object. In an exemplary embodiment, gripping device 133 is about 0.63 inches in length with a pitch of about 9 threads per inch.

In one embodiment, shaft 130 is generally cylindrical, but includes one or more flat outer surfaces 135. In a particular embodiment, second end 134 includes two rectangular flat, opposing surfaces which extend over the entire length of shaft 130, but terminate prior to gripping device 133. In an exemplary embodiment, the flat surfaces of shaft 130 are each about 1.25 inches in length.

In one embodiment, second end 134 of shaft 130 is configured to restrict shaft 130 from translating beyond a particular location with respect to the sleeve 110. In an exemplary embodiment, end cap 136 is located on or near second end 134, and is formed in a cylindrical configuration such that end cap 136 freely translates within the cylindrical portion of sleeve 110, but end cap 136 stops the translation of shaft 130, when end cap 136 impacts the flat inner surface of sleeve 110. End cap 136 limits the expansion of compressive device 140 to a certain point, so continued compression can be applied against the fracture. End cap 136 may be integral with shaft 130, welded onto shaft 130, or otherwise affixed to shaft 130.

With continued reference to FIG. 4, a wider diameter head 112 is located at the first end of sleeve 110. An exemplary diameter of head 112 is about 0.387 inches. Head 112 includes a recessed portion for receiving the hex head of a tool. One skilled in the art will appreciate that head 112 may be any configuration suitably configured to receive any suitable working tool. The recessed portion is about 0.10 inches in depth and about 0.198 inches wide. Head 112 (or any other portion of sleeve 110) may also include a ledge 114 (FIG. 5) for retaining compressive device 140 within sleeve 110. In various embodiments and as discussed herein, cap 20, described in '132 previously incorporated by reference, may be configured as sleeve 110 (or barrel) and any components of cap 20 described in '132 may be incorporated into bone screw 100.

A second end of sleeve 110 includes an opening 116 which receives shaft 130 such that shaft 130 is able to at least partially move within sleeve 110, with minimal or no movement of sleeve 110. As discussed above, in one embodiment, the inner surface of sleeve 110 is generally cylindrical, but the inside surface also includes two rectangular flat, opposing surfaces which extend along a portion of the length of sleeve 110. In an exemplary embodiment, the overall sleeve 110 is about 1.85 inches long, about 0.22 inches outer diameter, and about 0.161 inner diameter with a reduced distance between the flat surfaces of about 0.14 inches with the flat surfaces of sleeve 110 being each about 0.545 inches in length.

In one embodiment, and with respect to FIG. 5, a compressive device 140 exists between sleeve 110 and shaft 130 such that compressive device 140 exerts a force directly or indirectly against shaft 130. Compressive device 140 may include, for example, a spring or any other element which exerts a force and/or bears a load. In one embodiment, compressive device 140 is located inside sleeve 110 (as discussed above). In a particular embodiment, compressive device 140 is a spring having about 10 mm of extension. As such, compressive device 140 allows about 10 mm of compression before sleeve head 112 is no longer held against the cortex. Compressive device 140 may be suitably affixed to sleeve 110 and shaft 130 in any manner known in the art. In an exemplary embodiment, first end of compressive device 140 includes a larger diameter coil which sits upon ledge 114 of head 112, thereby restricting or minimizing translation of compressive device 140 within sleeve 110. The larger diameter coil may also be further retained by a C-clip 1014 or laser welding to sleeve 110 (e.g., at any location within the first end). Clip 1014 may be seated in head 112 preventing the internal components (e.g. the shaft, end cap, and/or spring) from protruding or exiting the distal end of sleeve 110 and/or head 112.

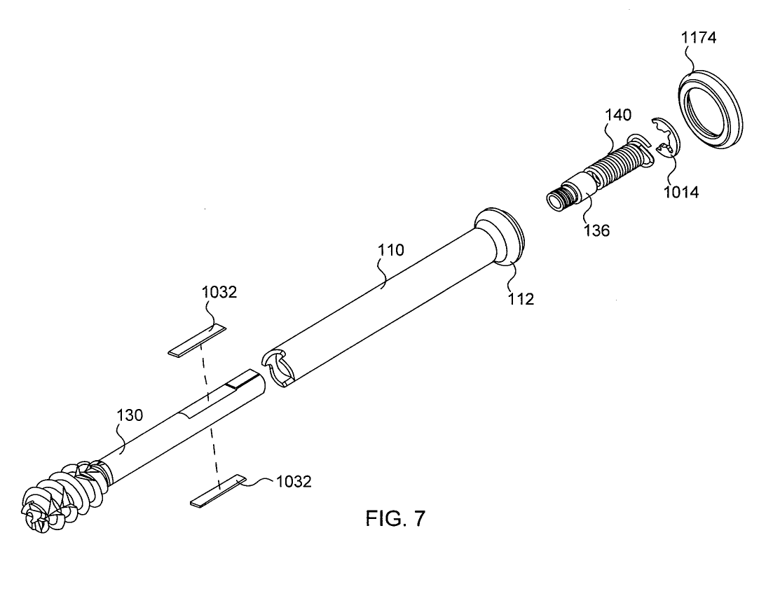

With reference to FIG. 7, and in accordance with various embodiments of the present invention, the bone screw may include one or more bushings 1032. Bushing 1032 may be located longitudinally along the exterior surface of the shaft 130. Bushing 1032 may be any shape, size, material, and configuration to provide low friction guidance between shaft 130 and sleeve 110. For example, bushing 1032 may be a rectangular flat material attached to the body of shaft 130, situated longitudinally along shaft's 130 exterior surface. In another example, bushing 1032 may be a cylindrically shaped material configured to attach around the circumference of the exterior surface of shaft 130.

In various embodiments, bushing 1032 may be configured to rigidly attach to shaft 130. Any method known in the art may be used to perform this attachment, including, for example, adhesive, screws, or corresponding fitted features (e.g. slot and groove attachment.

Bushing 1032 may also be configured to engage with sleeve 110 to prevent or minimize shaft 130 from rotating relative to sleeve 110. Bushing 1032 may also be configured to engage with sleeve 110 to provide a bearing surface, allowing efficient longitudinal translation of shaft 130 relative to sleeve 110. In one embodiment, sleeve 110 may include grooves configured to receive bushing 1032. In another embodiment, sleeve 110 may include a longitudinal rib which may be received by a corresponding groove in bushing 1032. Moreover, bushing 1032 may be any size sufficient to provide sufficient engagement between shaft 130 and sleeve 110. For example, bushing 1032 may extend the entire length of shaft's 130 unthreaded surface. In one embodiment, bushing 1032 may cover only an area on the distal and/or proximal end of shaft 1032.

In various embodiments, bushing 1032 may comprise any compound sufficient to provide low friction guidance to the shaft, including for example, Polyether ether ketone (PEEK); polyoxymethylene; Nylon; polytetrafluoroethylene; and/or any other compound sufficient to provide low friction guidance to the shaft.

In various other embodiments, second end of compressive device 140 may include a tang 142. Tang 142 may extend longitudinally from the perimeter of the end coil. Tang 142 may be crimped into a hole in shaft 130, laser welded to the end of shaft 130 and/or any other means for attaching tang 142 to shaft 130. In other embodiments, shaft 130 may abut compressive device 140, compressive device 140 may receive shaft 130 within its coils, or compressive device 140 may abut a component attached to shaft 130. For example, compressive device 140 may be a separate component suitably joined (e.g., welded, glued, molded) to shaft 130 and/or end cap 136.

Furthermore, referring to FIG. 7, end cap 136 may be a cylinder with threads on its distal end and a spring engagement surface on its proximal end. The threaded distal end of end cap 136 may be configured to be threaded into the proximal end of shaft 130 allowing the two to mate.

Locating compressive device 140 inside sleeve 110 is significantly advantageous because the compressive device is fully or partially protected from bone growth over and between the coils which may limit or destroy the functionality of the spring. Similarly, a re-absorbable material is not needed to be inserted between the coils in order to delay the compressive action of the spring. In other words, upon insertion, compressive device 140 is able to provide immediate and subsequent compression. Moreover, because shaft 130 and sleeve 110 rotate along with compressive device 140, bone screw device 100 may be inserted or removed with minimal or no torque or unraveling of compressive device 140.

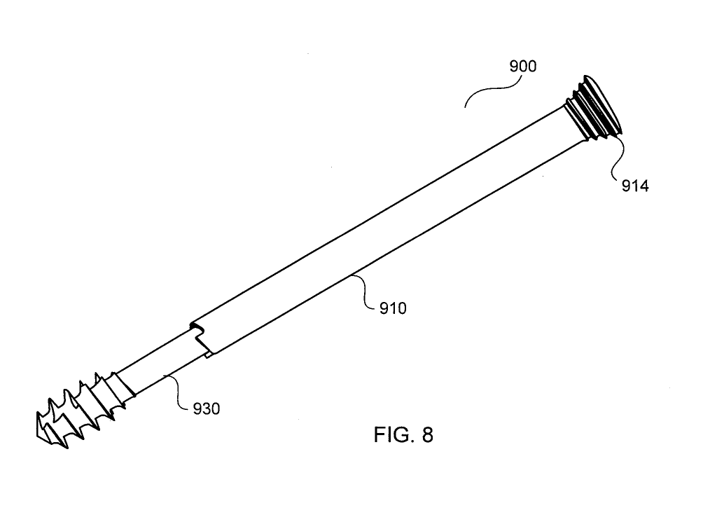

In an exemplary embodiment, the shaft of the bone screw device may be configured in a fully or partially extended position relative to the sleeve of the bone screw device before engaging the bone. For example, FIG. 8 illustrates bone screw device 900 having shaft 930 extended relative to sleeve 910. In an exemplary embodiment, the bone screw is configured in an extended position and then inserted through a first bone portion into a second bone portion.

In embodiments in which the bone screw comprises a compressive device (such as compressive device 140 illustrated in FIG. 5), extension of the bone screw causes the compressive device to expand and the tension of the compressive device to increase. When the bone screw is inserted through the first bone portion and into a second bone portion, contraction of the compressive device causes the first and second bone portions to compress.

The shaft may be extended using any known or hereinafter devised device, system or method. For example, FIG. 8 illustrates bone screw device 900 having longitudinal opening 922. Driver 923 is insertable into longitudinal opening 922 to push shaft 930 to a fully or partially extended position. In an embodiment, the longitudinal opening and driver may be the same shape (e.g., hex-shaped) to facilitate mating. However, any suitable shape may be used. In embodiments of the bone screw comprising a compressive device, the driver may extend through the center of the compressive device to contact the shaft. Extension of the shaft relative to the sleeve may occur with or without rotation of the compressive device. The extension of the shaft may occur prior to, during, or after the device is inserted into another object (e.g., plate, bone, etc).

In an exemplary embodiment, the driver may be configured to removably attach to the bone screw in order to maintain the bone screw in an extended position. For example, driver 923 may comprise attachment means 927 operable to be removably secured within longitudinal opening 922. Any suitable attachment means may be used. For example, the driver and/or bone screw may comprise one or more protrusions corresponding to recesses in the other component to allow the driver to be snapped, pressed or otherwise coupled together.

The user may position driver 923 within longitudinal opening 922 until gripping device 933 contacts a bone. Driver 923 may then be used to torque gripping device 933 into the bone. Alternatively, driver 923 may be removed and another suitable instrument may be used to screw gripping device 933 into the bone.

In an exemplary embodiment, the bone screw is not cannulated and may be inserted directly into a bone without a guide wire. In other embodiments, the bone screw may be cannulated.

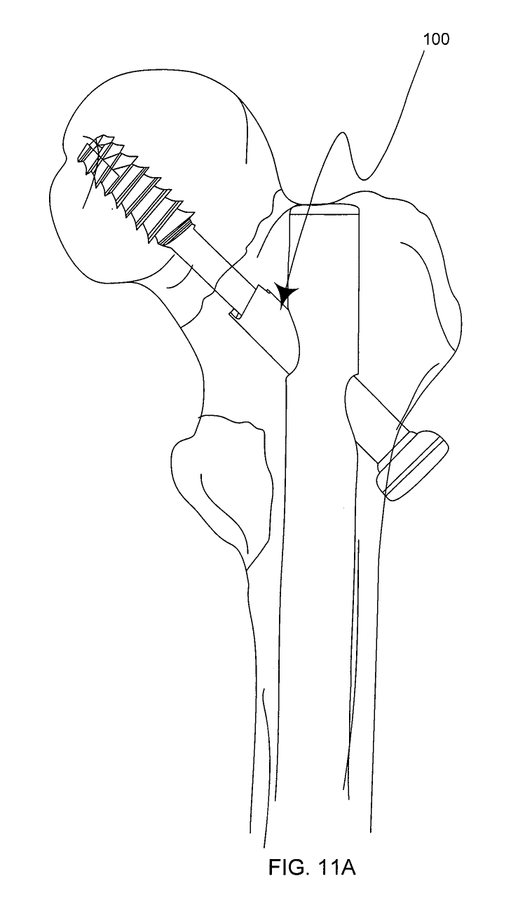

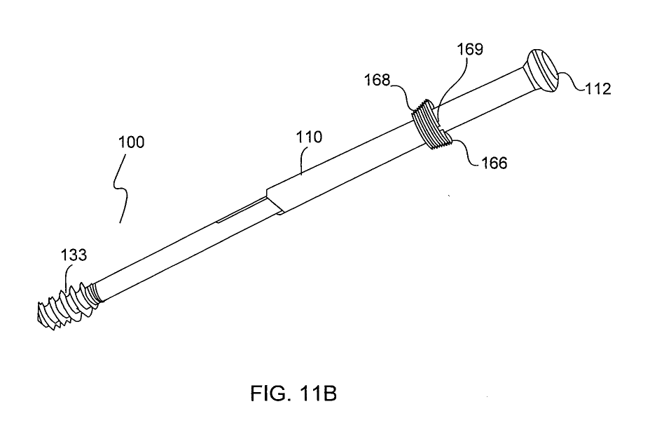

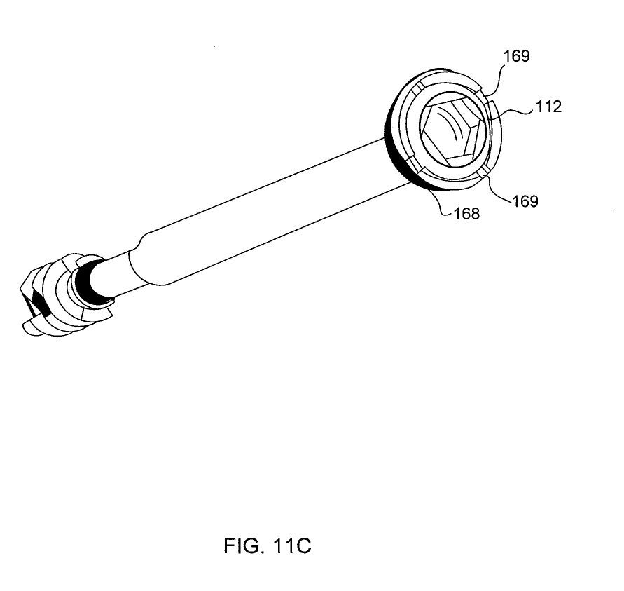

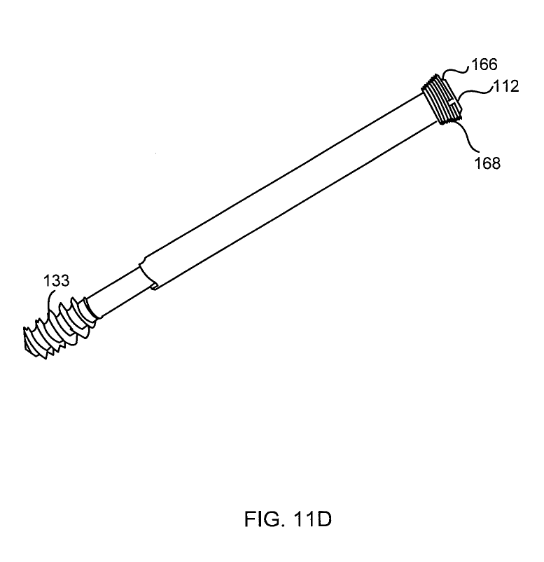

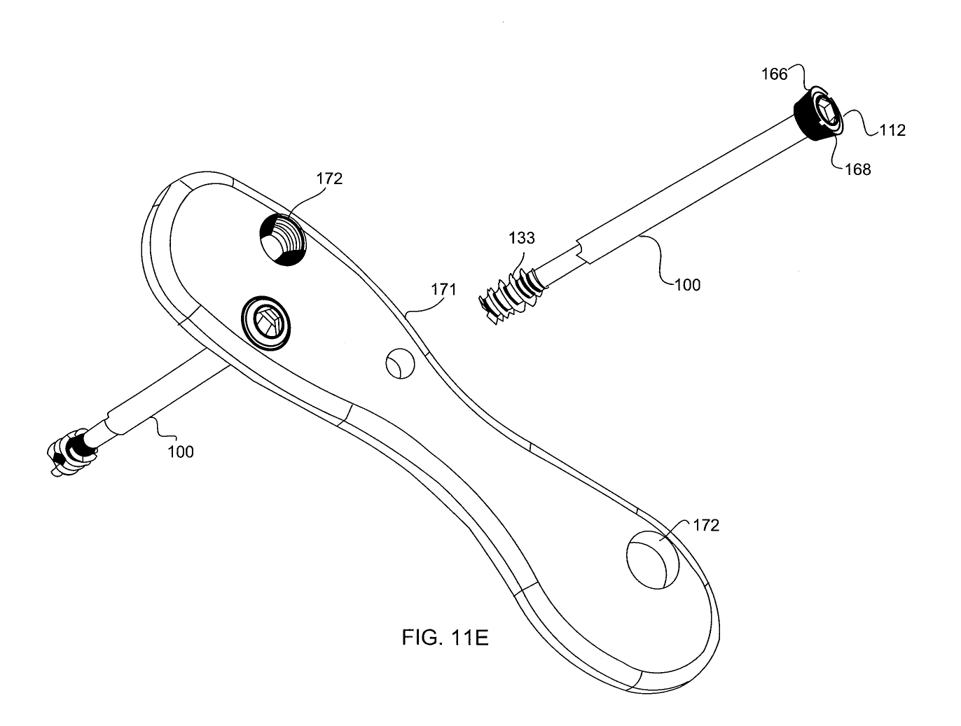

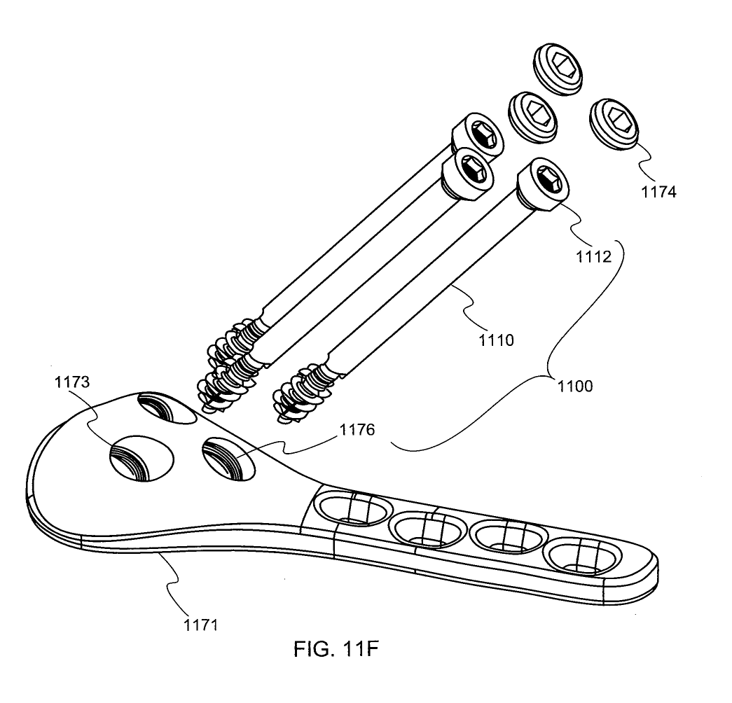

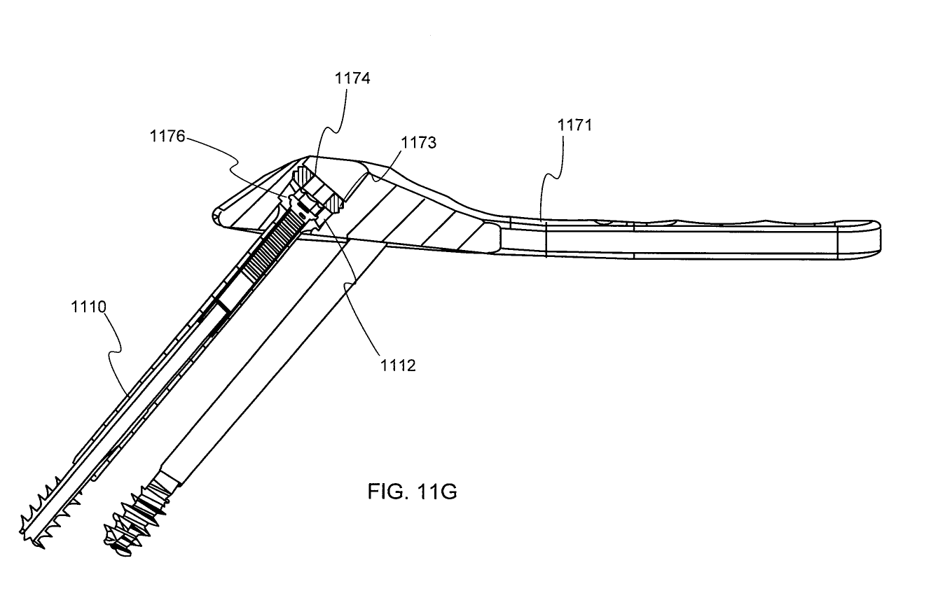

In an exemplary embodiment, the distal end of the bone screw may comprise a device for coupling the bone screw to a stabilization device, such as a locking plate. For example, FIGS. 7 and 8 illustrate bone screw 900 having ledge 914 for engaging a bone fixation device, such as locking plate 171 (illustrated in FIG. 11E). In an exemplary embodiment (and as illustrated in FIGS. 7 and 8), the ledge may comprise a gripping means, such as threads, to mate with corresponding threads on a stabilization device.

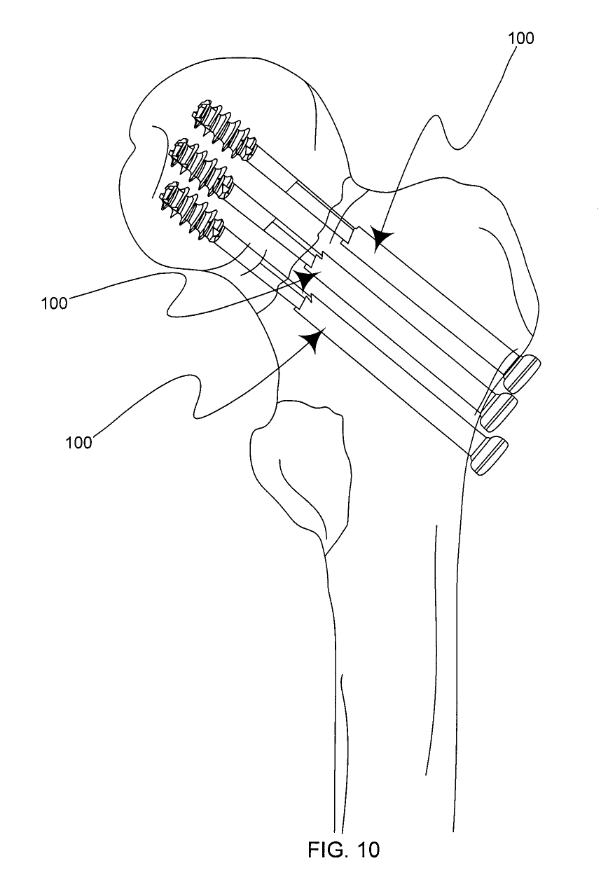

Multiple bone screws 100 of the present invention may also be used for rotational stability. For example, as set forth in FIG. 10, more than one bone screw (e.g., three) may be used to maintain compression and provide rotational stability in a fracture within the head of the femur bone.

Bone screw 100 of the present invention may be used in place of any existing bone screw, or any existing component of a product that performs a similar function as a bone screw. With respect to FIG. 11A, bone screw 100 is used in association with an intermedulary rod for additional support and stability.

A bone screw may also be configured for use with other bone stabilization devices, such as locking plates.

For example, a bone screw system may comprise an adapter operable to threadably mate with a stabilization device. An adapter may be any component, system or method which permits coupling of a bone screw with a bone stabilization device. In an embodiment, an adapter may be configured to restrict movement of a bone screw to a desired trajectory. FIG. 11B illustrates an exemplary embodiment of bone screw 100 comprising adapter 166.

In an embodiment, the adapter may be configured to couple to the head of a bone screw. Any known or hereinafter component, structure or method may be used to achieve coupling. For example, adapter 166 may comprise lip 169 having one or more notches configured to snap, screw or otherwise mate adapter 166 with retaining ring 170 located on head 112. FIGS. 11C & 11D further illustrate adapter 166 coupled to head 112. It will be understood that although a lip and retaining ring are used to couple the adapter and head in the illustrated embodiments, any suitable coupling structure or device, such as threads, snapping mechanisms and/or the like may be used.

In an embodiment, adapter 166 fits over sleeve 110 and is operable to slide along the length of the sleeve. Moreover, the hole within the adapter may be oriented perpendicular relative to the adapter or at any desired angle, so as to restrict movement of the bone screw to a desired trajectory.

As mentioned above, adapter 166 may be configured to couple with a stabilization device. Any known or hereinafter coupling component, device, structure or method such as notches, snapping mechanisms, and/or the like may be used. For example, FIG. 11B illustrates the outwardly-facing surface of adapter 166 comprising a plurality of threads 168. Threads 168 may be configured to couple with corresponding threads located on a stabilization device. For example, FIG. 11E illustrates a stabilization device 171 (in this case, a locking plate), comprising a plurality of holes 172. Holes 172 are threaded to permit coupling with threads 168 of adapter 166. It will be understood that when adapter 166 is mated with head 112 and coupled to locking plate 171, bone screw 100 may rotate but may not translate.

In various embodiments, the bone screw may be secured within the locking plate without rotation. For example, in an exemplary embodiment, a bone screw does not comprise (or has minimal) threads on the distal end, but still operably couples with a stabilization device. For example, FIGS. 11F & 11G illustrate bone screw 1100 having distal end 1112. The surface of distal end 1112 is substantially smooth and has a wider diameter than the body of sleeve 1110 to allow it to mate with counter-bore 1173 of locking plate 1171. Set screw 1174 mates with threads 1176 of locking plate 1171 to hold bone screw 1100 in place. In an exemplary embodiment, the bone screw does not fully or partially extend until after insertion into the locking plate.

As shown in FIGS. 11F & 11G, in an exemplary embodiment, the holes within the stabilization device may be oriented at a particular angle of entry, so as to restrict movement of the bone screw to a specific trajectory.

As with the other components of the present invention, including but not limited to the sleeve, the shaft and the bushings, the adapter may comprise any suitable physiologically acceptable material such as stainless steel, titanium, titanium alloy and/or PEEK material.

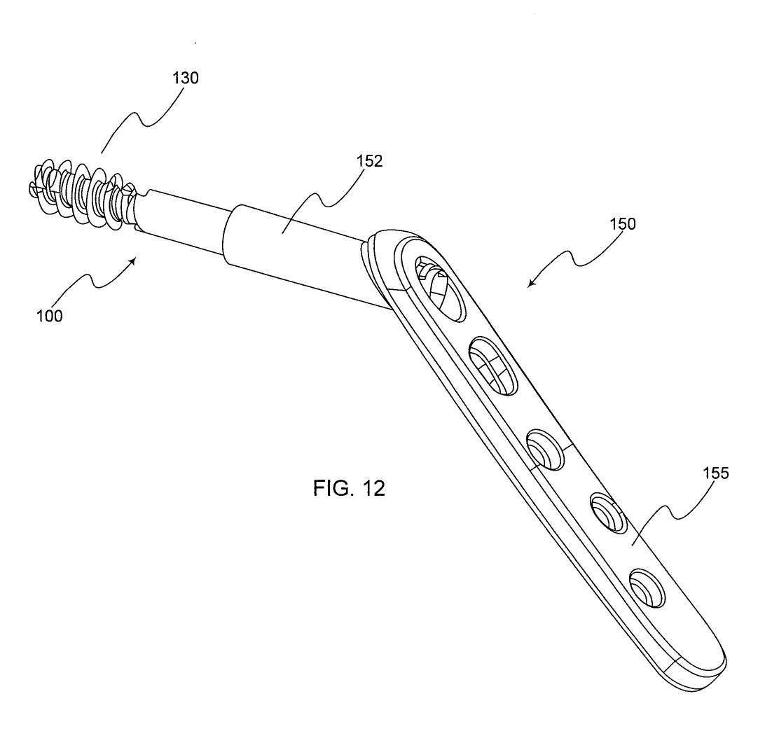

With respect to FIG. 12, bone screw 100 is incorporated into a compression/dynamic hip screw system 150 which may be used on, for example, a proximal femur fracture. An exemplary hip screw system 150 may include any combination of the various compression hip screw plates and nails manufactured by Smith & Nephew. In one embodiment, bone screw 100 is received into barrel 152 of hip screw system 150 in place of the standard bone screw which is typically received into barrel 152. Barrel 152 may or may not include an additional compressive device 140. In another embodiment, barrel 152 may act as a second sleeve 110, thereby adding to the available translation of shaft 130. In other words, shaft 130 translates within sleeve 110, and sleeve 110 itself may translate within barrel 152 before hip screw system 150 protrudes from the bone. In a further embodiment, sleeve 110 is affixed directly to plate 155, so a barrel is not needed.

Hip screw system 150 (with standard plate 155 and cortical bone screws) is inserted as is known in the art, and the features of the present invention incorporated into hip screw system 150 provide additional benefits by minimizing or preventing the device from protruding beyond the bone, and by maintaining an additional amount of compression across the fracture during fracture collapse. A T-Handle may be used to rotate bone screw 100 into the bone. One skilled in the art will appreciate that bone screw 100 may replace or supplement any of the screws (e.g., cortical bone screws, medial fragment screws and/or main bone screw) typically used in association with hip screw system 150.

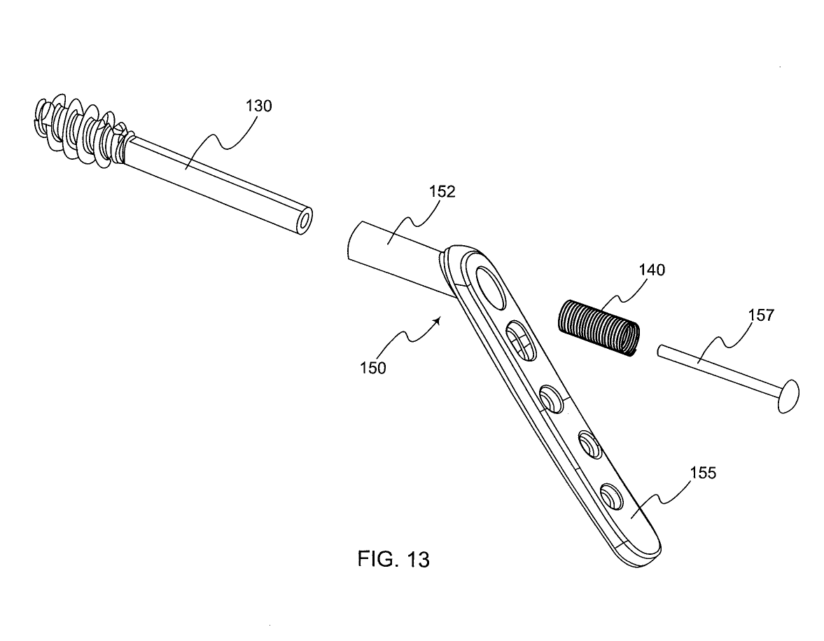

FIG. 13 shows another embodiment of hip screw system 150, wherein shaft 130 is received directly into barrel 152 of existing hip screw system 150, without the need for a separate sleeve 110. A standard barrel 152 may be used or a longer opening formed within barrel 152 to allow shaft 130 greater translation within barrel 152. Barrel 152 may also include any of the features and functions described above with respect to sleeve 110. For example, barrel 152 may include one or more flat inner portions to complement flat portion 135 of shaft 130, a ledge 114 to hold a wider diameter spring, etc. Any of the hip screw systems may or may not incorporate a compressive device 140 inside sleeve 110 or barrel 152. Without compressive device 140, barrel 152 and/or sleeve 110 is still configured to allow shaft 130 to collapse within barrel 152 and/or sleeve 110, as discussed above.

Compression screw 157 is inserted through plate 155, through barrel 152 and into shaft 130. Upon rotating or translating compression screw 157 through barrel 152, the head of compression screw 157 engages (or abuts) a recessed portion of plate 155 and/or a recessed portion of barrel 152. Upon continuing to rotate compression screw 157, shaft 130 is “pulled” back into barrel 152, thereby causing further compression. In another embodiment, compression screw 157 is also received through compressive device 140 which itself resides in barrel 152 and/or sleeve 110. Upon receiving a weight bearing load, hip screw system 150 allows shaft 130 to translate with minimal or no protrusion of hip screw system 150 beyond the bone, and also, maintaining an additional amount of compression across the fracture during fracture collapse.

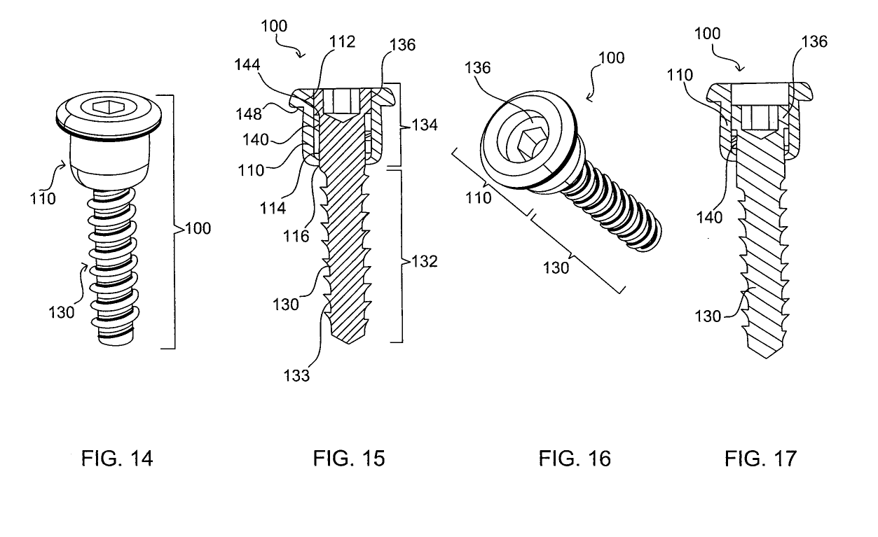

With respect to FIG. 14, another exemplary embodiment includes an improved screw 100 having a sleeve 110 and a shaft 130. In one embodiment, no additional elements exist between sleeve 110 and shaft 130, but in other embodiments (as discussed below in more detail and in FIGS. 15 and 17), a compressive device 140 (e.g. split washer) is located between sleeve 110 and shaft 130. In an exemplary embodiment, each of the elements sleeve 110, shaft 130, and compressive device 140 may be cannulated.

In one embodiment, with respect to FIG. 15, shaft 130 includes a first end 132 having a gripping device 133 and a second end 134. Gripping device 133 may include any structure and configuration for enabling shaft to enter and attach to an object. In one embodiment, gripping device includes a threaded surface thereon. The threaded surface may include cutting threads, mating threads, barbs, ribbed surface or any other surface configured to retain shaft 130 into an object. In an exemplary embodiment, gripping device 133 is about 0.63 inches in length with a pitch of about 14.3 threads per inch.

In one embodiment, second end 134 of shaft 130 is configured to restrict shaft 130 from translating beyond a particular location with respect to the sleeve 110. In an exemplary embodiment, end cap 136 is located on or near second end 134, and is formed in a cylindrical configuration such that end cap 136 freely translates within the cylindrical portion of sleeve 110, but end cap 136 stops the translation of shaft 130 when a bottom edge 144 of end cap 136 compresses compressive device 140 against a flat inner surface or ledge 114 of sleeve 110. An exemplary diameter of end cap 136 is about 0.22 inches.

End cap 136 includes a recessed portion for receiving the hex head of a tool.

One skilled in the art will appreciate that end cap 136 may be any configuration suitably configured to receive any suitable working tool. The recessed portion is about 0.1 inches in depth and about 0.12 inches wide. End cap 136 may include an axial length that is shorter than the axial length of the cylindrical portion of sleeve 110, such that end cap 136 may move within a range of distance capable of compressing, extending, and moving out of and into communication with compressive device 140 without exiting the chamber of the cylindrical portion of sleeve 110. This range of distance will ensure that compression from the fracture of an object, such as a bone, causing the shaft 130 to move towards the sleeve 110, will not cause the end cap 136 to exit the chamber within the cylindrical portion of sleeve 110, thereby avoiding a protruding end cap 136 from causing injury or inconvenience to a patient or other user of the screw 100. End cap 136 ensures the compression of compressive device 140 so continued compression can be applied against the fracture. End cap 136 may be integral with shaft 130, welded onto shaft 130, or otherwise affixed to shaft 130.

With continued reference to FIG. 15, a head 112 with a diameter wider than the end cap 136 may be located at the first end of sleeve 110. Alternatively, sleeve 110 may not include head 112. Rather, sleeve 110 may merely rest flush with an object, such as a bone, without having any ridge resting on the exterior surface of the object. An exemplary diameter of head 112 is about 0.4 inches. In one exemplary embodiment, head 112 includes a bottom edge 148 that abuts against the exterior surface of an object, such as a bone, bone plate 155 (FIG. 13), or barrel 152. In another embodiment, sleeve 110 may be formed as a barrel 152. Head 112 (or any other portion of sleeve 110) may also include a ledge 114, as previously identified, for retaining compressive device 140 within sleeve 110. Various components of the device described in '132 may be incorporated herein. For example, cap 20 described in '132 (as discussed above in other embodiments) may be configured as sleeve 110 (or barrel) and any components of cap 20 described in '132 may be incorporated into bone screw 100.

A second end of sleeve 110 includes an opening 116 which receives shaft 130 such that shaft 130 is able to at least partially move within sleeve 110, with minimal or no movement of sleeve 110. In an exemplary embodiment, the chamber within the cylindrical portion of the overall sleeve 110 is about 7 mm long, and the overall sleeve 110 is about 0.3 inches wide at the outer diameter, and about 0.21 inches wide at the inner diameter. In an exemplary embodiment, the overall end cap 136 located within the chamber of the cylindrical portion of sleeve 110 is about 2.5 mm long and about 0.21 inches wide at the outer diameter.

In one embodiment, and with respect to FIGS. 16 and 17, a compressive device 140 exists between sleeve 110 and shaft 130 such that compressive device 140 exerts a force directly or indirectly against shaft 130. Compressive device 140 may include, for example, a spring, split washer, or any other element which exerts a force and/or bears a load. In one embodiment, compressive device 140 is located inside sleeve 110 (as discussed above). In a particular embodiment, compressive device 140 is a split washer having about 1 mm of expansion and compression formed in a helical shape. As such, compressive device 140 allows about 1 mm of compression before end cap 136 fully compresses compressive device 140, or, conversely, about 1 mm of extension before end cap 136 fully relaxes compressive device 140. When end cap merely rests against relaxed and fully extended compressive device 140, there is approximately 1 mm of distance between the outer surface of end cap 136 and the outer surface of sleeve head 112. Compressive device 140 is shown either relaxed and in contact with end cap 136 or at least partially compressed in FIG. 17 such that sleeve 110 and shaft 130 are at least in contact with or indirectly exerting force against each other. In its partially compressed state, compressive device 140 permits end cap 136 to recede within the cavity or chamber formed within the cylindrical portion of sleeve 110, as shown in FIG. 16.

In accordance with an exemplary embodiment, a bone screw system may be used to deliver treatment to a desired location. The treatment may be delivered by any bone screw system, wherein the bone screw system may comprise any composition, device or structure that will facilitate the fixation and/or provide support to bones. The treatment may comprise medications (such as bone growth stimulation drugs or structures), adhesives, implants, fasteners, ligaments, tendons, antibiotics and suturing materials. In one embodiment, a bondable material may be delivered to the bone to facilitate the joining of bone fragments. For example, the materials disclosed in U.S. Pat. No. 7,217,290 entitled “SURGICAL DEVICES CONTAINING A HEAT BONDABLE MATERIAL WITH A THERAPEUTIC AGENT,” (the '290 Patent) which is herein incorporated by reference in its entirety, may be delivered to a region of interest using the bone screw system disclosed herein.

In one embodiment, a portion or all of the surface of the bone screw system may be partially or fully coated in the medication. In another embodiment, specific components of the bone screw device may be configured to deliver the medication, such as the shaft, sleeve, and/or the bushings. For example, the treatment may be delivered to the bone through the center of one or more of the screw's components (e.g. the shaft, sleeve, threads, compression device, etc).

A desired location for the medication may be any position on or within one or more bones. It will be understood that the present system and method may be used in connection with any type of bone, such as a clavicle, pelvis, humerus, tibia, ulna, and/or the like. In one embodiment, a bone screw system may be used to deliver treatment to the interior of a bone. For example, the bone screw system may be used deliver treatment via an intermedullary canal.

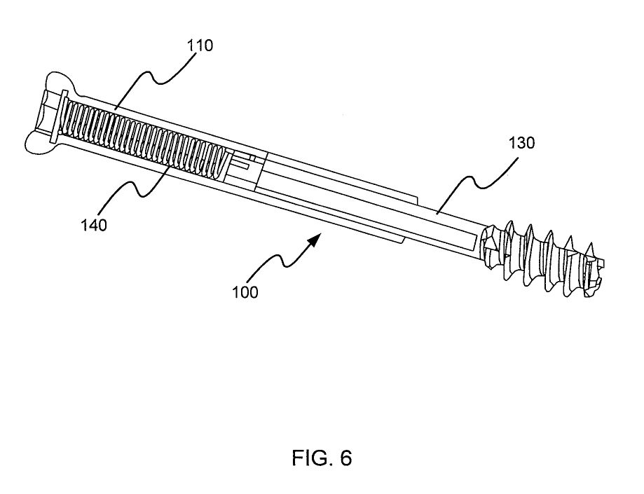

Having described exemplary components of the invention, exemplary methods for inserting bone screw 100 will now be described. An exemplary method for inserting bone screw 100 comprises drilling a bore hole into the two objects (e.g., two pieces of the fractured bone) which are to be compressed together. In an exemplary method used in conjunction with the bone screw 100 described with reference to FIGS. 14 through 17, one or more coaxial bore holes may be drilled, having different diameters and depths in order to accommodate the insertion of a sleeve 110 having a wider diameter and shorter depth than a shaft 130 having a narrower diameter and longer depth. A guide rod may be inserted into the bore hole, then bone screw 100 may be inserted over the guide rod. Either head 112 (FIGS. 4 through 9) or end cap 136 (FIGS. 14 through 17), depending upon the embodiment employed, of bone screw 100 is then rotated (e.g. using a drill, hex head driver, or other suitable device) into and through the proximal bone portion or fragment. Head 132 of shaft 130 then enters the distal bone portion or fragment. When sleeve 110 impacts or sits flush against the surface of the proximal bone portion or fragment (or against a plate placed over the bone portion or fragment), either head 112 (FIGS. 4 through 9) or end cap 136 (FIGS. 14 through 17), depending upon the embodiment employed, of sleeve 110 continues to rotate, but sleeve 110 no longer translates into the bone. However, the rotation of sleeve 110 or end cap 136, depending upon the embodiment employed, continues to advance shaft 130 further into the distal bone portion or fragment because threads of gripping device 133 move shaft 130 forward. Such continued translation and penetration of shaft 130 into the distal bone portion or fragment also extends compressive device 140 (as best shown in FIG. 6) or compresses compressive device 140 (as best shown in FIGS. 16 and 17), depending upon the embodiment employed. In other words, the continued advance of shaft 130 causes compressive device 140 to stretch beyond its relaxed condition (as shown in FIG. 6) or compress from its relaxed helical condition towards a flat condition (as shown in FIG. 17). After the bone screw is appropriately inserted, the guide rods are removed.

One skilled in the art will appreciate that shaft 130 may penetrate into the distal bone portion or fragment any desired partial or full distance, and thus, extend or compress, as applicable, compressive device 140 to any desired partial or full extension, compression, or force. One skilled in the art will appreciate that any “rotational insertion” discussed herein may alternatively or additionally include other means for insertion such as, for example, a direct translation using a hammer to force the shaft and/or sleeve into the bone.

After insertion of bone screw 100, compressive device 140 exerts force against sleeve 110 and shaft 130, thereby forcing the components either toward or away from one another, depending upon the embodiment employed. Such force helps to maintain the compressive load at the union of the fracture. As additional compression is exerted on the load in a fracture collapse (e.g., from weight bearing), the bone is compressed closer together, so force may be reduced. However, the present invention either collapses or expands, as applicable, in association with the fracture collapse to substantially minimize or prevent sleeve head 112 of bone screw 100 (FIGS. 4 through 9) from protruding beyond the bone or to substantially minimize or prevent end cap 136 of bone screw 100 (FIGS. 14 through 17) from protruding beyond the chamber within the cylindrical portion of head 112. In other words, sleeve head 112 is substantially maintained against the lateral cortex, while compressive device 140 maintains compression across the fracture during fracture collapse. That is, as the bone portions or fragments undergo stress relaxation, bone screw 100 similarly relaxes, while continuing to hold the portions or fragments together. As such, bone screw 100 continues to accommodate the stress relaxation of the bone portions or fragments until the fracture therebetween has significantly or completely healed.

As discussed above, in one embodiment, compressive device 140 is a spring having about 10 mm of extension. As such, the spring allows about 10 mm of compression before shaft 130 impacts sleeve 110 so that sleeve head 112 is forced away from the cortex. Sleeve head 112 may be maintained against the lateral cortex until a sufficient amount of force no longer exists within compressive device 140, then bone screw 100 may simply act as a traditional bone screw.

As also discussed above, in another embodiment, compressive device 140 is a split washer having about 1 mm of compression. As such, the split washer allows about 1 mm of extension before end cap 136 of shaft 130 moves away from compressive device 140 in a direction towards the exit of the chamber of the cylindrical portion of sleeve 110. Unlike the embodiment discussed with reference to FIGS. 4 through 9, the embodiment discussed with reference to FIGS. 14 through 17 provides an additional advantage of permitting the shaft 130 to move fully exit sleeve 110 without ever forcing sleeve 110 or sleeve head 112 away from the cortex. As with the embodiment discussed with reference to FIGS. 4 through 9, the embodiment discussed with reference to FIGS. 14 through 17 provides a sleeve head 112 that may be maintained against the lateral cortex until a sufficient amount of force no longer exists within compressive device 140, then bone screw 100 may simply act as a traditional bone screw.

In an embodiment, a system of the present invention may comprise a stabilization device operable to permit a user to create a hole for insertion one or more fasteners at any desired location on, within and/or around the stabilization device. A stabilization device may be any device or structure that suitably provides stabilization to one or more bone fragments. For example, a stabilization device may comprise a bone plate, locking plate, intermedullary rod, artificial vertebrae, and/or the like. A fastener may generally comprise any mechanism for securing a stabilization device to a bone, including for example a cap, bone screw, lagscrew, lagwire, pin, wire and/or the like.

In an exemplary embodiment, a stabilization device may comprise at least a portion of penetrable material which suitably allows a user to drill one or more holes for insertion of a fastener at a desired location, while maintaining a partial or complete sterile environment. In some embodiments, the penetrable material may be non-metallic, moldable, and/or inert such that any shavings produced while drilling the holes will not be harmful to the patient. Suitable materials may include, for example, plastics such as polyetheretherketone (PEEK). The material may be any desired hardness. For example, the material may be more, less or substantially the same hardness as a bone. Moreover, the material may be embedded with carbon fibers to create the desired material strength. It will be understood that any material which suitably permits a hole to be created for insertion of a fastener (using, for example, manual or automatic power) may be used.

In various embodiments, all or substantially all of the stabilization device may comprise a penetrable material. However, in other embodiments, the stabilization device may comprise one or more portions of penetrable material and/or conventional materials. Conventional materials include, for example, titanium, stainless steel and/or titanium alloy. For example, in an embodiment, a stabilization device may comprise a bone plate having a central portion comprising a penetrable material and a peripheral portion comprising a conventional material. In another embodiment, comprised of a conventional material that is surrounded all or in part by a penetrable material. It will be understood that a stabilization device may comprise any desirable combination penetrable and conventional material portions and fall within the scope of the present invention.

In an embodiment, the stabilization device does not comprise any pre-existing holes for insertion of fasteners. Rather, a user determines a desired entry point location and angle of entry of a fastener and then creates one or more holes manually or using automatic power, such as a drill. In other embodiments, the stabilization device may comprise one or more pre-existing holes operable to couple the stabilization device to the bone with wires, screws, and/or the like. The user may then create one or more additional holes in the stabilization device at desired locations and angles for insertion of additional fasteners. It will be understood that a stabilization device of the present invention may comprise any number of pre-existing and/or user-created holes and fall within the scope of the present invention.

The stabilization device may also include a “kit” of other items which are used in association with the stabilization device. For example, the kit may include a template, tap and/or a router to allow the physician to configure the device to one of many template options, or customize the device to any desired shape or topography.

In accordance with an exemplary method, a stabilization device may be installed onto a patient by performing the steps of: selecting a stabilization device having at least a portion of penetrable material; positioning the stabilization device at a desired location on, adjacent to, or within a bone; selecting one or more entry point locations and angles of entry for a fastener; creating a hole within the penetrable material at the desired location and angle of entry using manual or automatic power; and inserting a fastener into the hole to couple the stabilization device to the bone. The method discussed herein may optionally include the additional step of washing away any shavings produced while creating the hole.

The present invention is described herein in connection with the fixation of bone fractures; however, one skilled in the art will appreciate that the lagwire or bone screw system and method described herein may also be used for changing, maintaining, reducing or expanding the distance between objects, object portions, or surfaces, compressing objects or object portions together, or providing pressure to surfaces. For example, the present invention may be used to repair wood products, tree limb damage, breaks in supports or columns, cracks in sculptures or buildings, fractures in sections of concrete or other building materials, cracks or breaks in car parts and/or the like.

In the foregoing specification, the invention has been described with reference to specific embodiments. Various modifications and changes can be made, however, without departing from the scope of the present invention as set forth in the claims below. The specification and figures are to be regarded in an illustrative manner, rather than a restrictive one, and all such modifications are intended to be included within the scope of present invention. Accordingly, the scope of the invention should be determined by the appended claims and their legal equivalents, rather than by the examples given above. For example, the steps recited in any of the method or process claims may be executed in any order and are not limited to the order presented in the claims.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the invention. The scope of the invention is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the specification or claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. All structural, chemical, and functional equivalents to the elements of the above-described exemplary embodiments that are known to those of ordinary skill in the art are expressly incorporated herein by reference and are intended to be encompassed by the present claims. Further, a list of elements does not include only those elements but may include other elements not expressly listed or inherent to such process, method, article, or apparatus.

We claim:

1. A system for compressing a first bone fragment and a second bone fragment, said system comprising:
a sleeve comprising a proximal sleeve end, a distal sleeve end, and a sleeve body located between said proximal sleeve end and said distal sleeve end, wherein said proximal sleeve end comprises threads;
a shaft comprising a proximal shaft end, a distal shaft end, and a shaft body, wherein said proximal shaft end is reciprocally received within said sleeve, wherein said proximal shaft end is retained between said distal sleeve end and said proximal sleeve end, wherein said proximal shaft end comprises shaft threads configured to mate with said second bone fragment; and
a compression device located within said sleeve, and said compression device is engaged between said proximal shaft end of said shaft and said proximal sleeve end of said sleeve.

2. The system of claim 1, wherein said threads mate with at least one of threads on a bone plate and said first bone fragment causing said sleeve to be anchored to at least one of said bone plate and said first bone fragment.

3. The system of claim 1, wherein said distal sleeve end and said sleeve body comprise threads.

4. The system of claim 1, wherein said proximal sleeve end comprises a head which is larger in diameter than said sleeve body, wherein said threads are located on said head and are configured to mate with threads on a bone plate.

5. The system of claim 1 further comprising an extension tool configured to insert into a longitudinal opening on said proximal end of said sleeve, wherein said extension tool is configured to contact said shaft and translate said shaft at least partially out of said sleeve.

6. The system of claim 1, wherein at least one of said sleeve and said shaft includes a medicated coating.

7. The system of claim 6, wherein said medicated coating is an antibiotic.

8. The system of claim 1, wherein said shaft further includes a longitudinal bushing.

9. The system of claim 8, wherein at least one of said shaft, said sleeve, and said bushing comprises a peek material.

10. A method for preparing and inserting a bone screw into a first bone fragment and a second bone fragment, comprising:
locking said bone screw into an extended position by inserting a driver into a longitudinal opening of a sleeve to contact a shaft;
pushing a bone screw shaft at least partially out of said sleeve causing a compressive device contained within said sleeve to extend;
implanting said bone screw in said first bone fragment and said second bone fragment; and
unlocking said bone screw by removing said driver from said bone screw causing said compressive device to compress said first bone fragment and said second bone fragment.

11. The method of claim 10, further comprising securing a distal end of said bone screw to a bone plate attached to said first bone fragment.

12. The method of claim 10, further comprising delivering medication to said first bone fragment and said second bone fragment by a medicated coating on said bone screw.

13. The method of claim 10 further comprising securing a distal end of said bone screw to said first bone fragment.

14. The method of claim 10, wherein at least one of said shaft, said sleeve, and said bushing comprises a peek material.

15. A system for compressing a first bone fragment and a second bone fragment, said system comprising:
a sleeve comprising a proximal sleeve end, a distal sleeve end, and a sleeve body located between said proximal sleeve end and said distal sleeve end, wherein said proximal sleeve end comprises threads, wherein said proximal sleeve end comprises a head portion larger in diameter than at least one of said body or distal sleeve end;
a shaft comprising a proximal shaft end, a distal shaft end, and a shaft body, wherein said proximal shaft end is reciprocally received within said sleeve, wherein said proximal shaft end comprises shaft threads configured to mate with said second bone fragment, wherein said shaft includes a flat bushing mated to said shaft body in a longitudinal direction;
an endcap mated to said proximal shaft end, wherein said endcap retains said proximal shaft end between said proximal sleeve end and said distal sleeve end;
a compression device located within said sleeve, wherein a first end mates with said endcap, and a second end mates with said proximal sleeve end; and
a c-clip seated in said head portion configured to retain said compression device, said endcap, and said shaft.

16. The system of claim 15, wherein said threads mate with at least one of threads on a bone plate and said first bone fragment causing said sleeve to be anchored to at least one of said bone plate and said first bone fragment.

17. The system of claim 15, wherein said distal sleeve end, and said sleeve body comprise threads.

18. The system of claim 15, wherein said proximal sleeve end comprises a head which is larger in diameter than said sleeve body, wherein said threads are located on said head and are configured to mate with threads on a bone plate.

19. The system of claim 15 further comprising an extension tool configured to insert into a longitudinal opening on said proximal end of said sleeve, wherein said extension tool is configured to contact said shaft and translate said shaft at least partially out of said sleeve.

20. The system of claim 15, wherein one of at least said sleeve and said shaft includes a medicated coating.

21. The system of claim 20, wherein said medicated coating is an antibiotic.

22. The system of claim 15, wherein at least one of said shaft, said sleeve, and said bushing comprises a peek material.

23. A method for delivery of a treatment to the interior of a bone comprising:
inserting a bone screw into said bone, wherein said bone screw includes a passageway through a center of said bone screw and
delivering a treatment to the interior of said bone through said passageway in said bone screw.
